(19) United States
(12) Patent Application Publication
(10) Pub. No.: US 2017/0166944 A1
(43) Pub. Date: Jun. 15, 2017

(54) ISOLATED POLYNUCLEOTIDE INCLUDING PROMOTER REGION, HOST CELL INCLUDING THE SAME, AND METHOD OF EXPRESSING TARGET GENE USING THE HOST CELL

(71) Applicant: Samsung Electronics Co., Ltd., Suwon-si (KR)

(72) Inventors: Jiae Yun, Hwaseong -si (KR); Hongsoon Rhee, Suwon -si (KR)

(21) Appl. No.: 15/340,188

(22) Filed: Nov. 1, 2016

(30) Foreign Application Priority Data
Dec. 9, 2015 (KR) 10-2015-0175342

(51) Int. Cl. C12P 21/02; C12N 9/10; C12N 9/90; C12N 9/14; C12N 9/12

(52) U.S. Cl. CPC C12P 21/02; C12N 9/14; C12N 9/1205; C12N 9/90; C12N 9/1241; C12N 9/1051; C12Y 207/01002; C12Y 504/02002; C12Y 207/07009; C12Y 204/01012; C12Y 306/03034

(57) ABSTRACT

Provided is a recombinant polynucleotide including a promoter region derived from an acetic acid-producing bacterium and a polynucleotide sequence encoding a target protein operably linked to the promoter, a host cell including the same, and a method of expressing a target gene or protein using the host cell.

### CROSS-REFERENCE TO RELATED APPLICATION

This application claims the benefit of Korean Patent Application No. 10-2015-0175342, filed on Dec. 9, 2015, in the Korean Intellectual Property Office, the entire disclosure of which is hereby incorporated by reference.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 12,263 Byte ASCII (Text) file named “726012_ST25.TXT,” created on Nov. 1, 2016.

### BACKGROUND

1. Field

The present disclosure relates to isolated recombinant polynucleotide including a promoter region derived from an acetic acid-producing bacterium and a polynucleotide sequence encoding a target protein operably linked to the promoter, a host cell including the same, and a method of expressing a target gene using the host cell.

2. Description of the Related Art

A promoter is required for expression of a target gene in a microorganism. The promoter is a region of DNA where an RNA polymerase binds during the transcription of DNA into mRNA. The binding of RNA polymerase, or other transcription factors, to the promoter region and the subsequent level of mRNA expression is dependent upon the DNA base sequence and the length of the promoter. In other words, the promoter determines the expression level of the gene under given conditions. Therefore, to improve microorganisms as desired, a proper promoter capable of expressing a target gene at a desired expression level is needed.

Acetic acid bacteria generally refer to bacteria that produce acetic acid during fermentation for vinegar production. Acetic acid bacteria have the ability to produce many organic acids such as acetic acid, and thus, have been traditionally used in the food and beverage industry. Additionally, it was revealed that a vinegar film produced by the acetic acid bacteria during fermentation for vinegar production is composed of cellulose. Such microbial cellulose is highly sought after as it has many industrial applications. However, it is difficult to produce large quantities of the microbial cellulose due to a low production rate in acetic acid bacteria. Therefore, it is crucial to increase the production rate of microbial cellulose in acetic acid bacteria. This invention provides such a method and host cell to increase the production rate of microbial cellulose in acetic acid bacteria.

### SUMMARY

One aspect of the invention provides a recombinant polynucleotide comprising a promoter region having a nucleotide sequence comprising positions 421 to 450 of SEQ ID NO: 1, a nucleotide sequence comprising positions 248 to 273 of SEQ ID NO: 2, or a nucleotide sequence comprising SEQ ID NO: 3, and a polynucleotide sequence encoding a target protein operably linked to the promoter region.

Another aspect provides a host cell including the recombinant polynucleotide.

Still another aspect provides a method of expressing a target gene to produce a target protein using the host cell.

### BRIEF DESCRIPTION OF THE DRAWINGS

These and/or other aspects will become apparent and more readily appreciated from the following description of the exemplary embodiments, taken in conjunction with the accompanying drawing in which:

FIG. 1 shows results of a choramphenicol acetyltransferase (CAT) assay.

### DETAILED DESCRIPTION

One aspect of the invention provides a recombinant polynucleotide including a promoter region having a nucleotide sequence of positions 421 to 450 of SEQ ID NO: 1, a nucleotide sequence of positions 248 to 273 of SEQ ID NO: 2, or a nucleotide sequence of SEQ ID NO: 3, and a target gene polynucleotide sequence encoding a target protein.

The term “promoter”, as used herein, refers to a DNA region to which an RNA polymerase binds to initiate transcription of a gene operably linked thereto. The sequence of a promoter may be modified (e.g., mutated) by those skilled in the art, such that the resulting promoter has the same or similar activity. Thus, a promoter having a sequence homology of, for example, 70% or higher, 80% or higher, 90% or higher, or 95% or higher to the nucleotide sequence of positions 421 to 450 of SEQ ID NO: 1, the nucleotide sequence of positions 248 to 273 of SEQ ID NO: 2, or the nucleotide sequence of SEQ ID NO: 3 are included in the scope of the invention.

Additionally, a fragment including sequences having the promoter activity in the nucleotide sequence of positions 421 to 450 of SEQ ID NO: 1, the nucleotide sequence of positions 248 to 273 of SEQ ID NO: 2, or the nucleotide sequence of SEQ ID NO: 3, for example, sequences (hereinafter, referred to as “variant”) of a predicted transcription start site and −10 element may be also included in the scope of the present invention.

The promoter may comprise, consist essentially of, or consist of a nucleotide sequence of SEQ ID NO: 1, 2, or 3.

The recombinant polynucleotide may be a vector. In the recombinant polynucleotide, the target gene may be operably linked to the promoter region. The target gene may be operably linked downstream of the promoter region. The term “operably linked” as used herein means that a gene to be expressed is functionally linked to its control sequences so that expression of the gene is allowed. The vector may further include a replication origin, a promoter control site, a ribosome binding site, a transcription termination site, a selection marker, or a combination thereof, as well as the promoter or variant thereof and the target gene.

The term “vector”, is a term known to those of ordinary skill in the art and refers to a construct for transferring a nucleic acid into cells. The vector may include, for example, a plasmid or a vector derived from a virus. A “plasmid” refers to a circular, double-stranded DNA loop. Certain vectors are capable of autonomous replication in a host cell into which they are introduced (e.g., bacterial vectors having a bacterial origin of replication). Other vectors are integrated into the genome of a host cell upon introduction into the host cell, thereby being replicated along with the host genome. Moreover, certain vectors may direct expression of genes to which they are operatively linked. Such vectors are referred to herein as “expression vectors”. In general, expression vectors of utility in recombinant DNA techniques are often in the form of plasmids. The vector used herein may include, for example, a plasmid expression vector, a viral expression vector, and a viral vector capable of performing a function equivalent thereto.

The vectors may include one or more regulatory sequences, selected on the basis of the host cells to be used for expression, and are operatively linked to the nucleic acid sequence to be expressed. “Operatively linked” means that the nucleotide sequence of interest is linked to the regulatory sequence(s) in a manner which allows for expression of the nucleotide sequence in the host cell. The term “regulatory sequence” is intended to include promoters, enhancers and other expression control elements. Regulatory sequences include those which direct constitutive expression of a target nucleic acid in many types of host cells and those which direct expression of the target nucleic acid only in particular host cells (e.g., tissue-specific regulatory sequences). It will be appreciated by those skilled in the art that the design of the expression vector may depend upon factors such as choice of the host cell to be transformed, an expression level of a protein desired, or the like. The expression vector of the present invention may be introduced into the host cell to express the protein.

The plasmid may be a bacterial cloning vector. These cloning vectors may include a site that allows DNA fragments to be inserted, for example, a multiple cloning site or a polylinker having several commonly used restriction sites to which DNA fragments may be ligated. After the gene of interest is inserted, the plasmids are introduced into bacteria by transformation. These plasmids may include a selectable marker, usually, an antibiotic resistance gene, which confer on the bacteria an ability to survive and proliferate in a selective growth medium containing the particular antibiotics. The cells after transformation are exposed to the selective media, and only cells containing the plasmid may survive. In this way, the antibiotics act as a filter to select only the bacteria containing the plasmid DNA. The vector may also contain other marker genes or reporter genes to facilitate selection of plasmid with the cloned insert. Thereafter, bacteria containing the plasmid may be grown in large amounts, harvested, and then isolated using various methods of plasmid preparation. A plasmid cloning vector may be used to clone DNA fragments of about 15 kbp or shorter. The vector may be a commercially available vector, for example, a pBR322, pUC, or TOPO cloning vector.

The target gene may be any gene encoding a target protein heterologous (non-native) to the promoter. The target gene may encode a protein involved in cellulose production, for example, synthesis. The gene encoding the target product may be a gene encoding permease, glucose kinase (GLK), phosphoglucomutase (PGM), UDP-glucose pyrophosphorylase (UGP), or cellulose synthase (CS). The gene encoding the target product may be an E. coli (Ec) glcP gene, a Zymomonas mobilis (Zm) glk gene, a Komagataeibacter xylinus (Kx) pgm gene, an E. coli (Ec) galU gene, a Xanthomonas campestris (Xc) UGP gene, or a Komagataeibacter xylinus (Kx) bcsABCD gene.

The recombinant polynucleotide may be produced by any technique. The term “recombinant” in this respect means that the polynucleotide is produced by genetic engineering, and is non-naturally occurring. The recombinant polynucleotide maybe synthetic, or semisynthetic.

Under aerobic conditions, the recombinant polynucleotide may increase expression of the operably linked target gene by 0.5 time or higher, for example, 1 time, 1.2 times, 1.3 times, or 1.4 times or higher, compared to that of the gene operably linked to a tac promoter, based on mRNA or protein level.

Another aspect provides a host cell including the recombinant polynucleotide.

The host cell may be an acetic acid-producing bacterium. The host cell may be a cell of the family Acetobacteraceae. The cell of the family Acetobacteraceae may be a cell of the genus Komagataibacter, the genus Gluconacetobacter, the genus Acetobacter, or the genus Gluconobacter. The host cell may be a Komagataibacter xylinus (also called “Acetobacter xylinum”) cell. The host cell may be a recombinant host cell.

In the host cell, the recombinant polynucleotide may be a vector. In the polynucleotide, the target gene may be operably linked to the promoter region. The target gene may encode the target protein. The target gene may encode a protein involved in cellulose production, for example, cellulose synthesis. The gene encoding the target product may be a gene encoding permease, glucose kinase (GLK), phosphoglucomutase (PGM), UDP-glucose pyrophosphorylase (UGP), or cellulose synthase (CS). The gene encoding the target product may be an E. coli (Ec) glcP gene, a Zymomonas mobilis (Zm) glk gene, a Komagataeibacter xylinus (Kx) pgm gene, an E. coli (Ec) galU gene, a Xanthomonas campestris (Xc) UGP gene, or a Komagataeibacter xylinus (Kx) bcsABCD gene.

The vector may be introduced into the host cell to clone the target gene or to express the target gene. The introduction of the vector may be performed by applying appropriate standard techniques known in the art, depending on the host cell, for example, by electroporation, heat-shock, calcium phosphate (CaPO₄) precipitation, calcium chloride (CaCl₂) precipitation, microinjection, a polyethylene glycol (PEG) method, a DEAE-dextran method, a cationic liposome method, a lithium acetate-DMSO method, or a combination thereof.

In the vector introduced to the host cell, a gene encoding the target protein may be operably linked downstream of the promoter. The vector may further include a replication origin, a promoter control site, a ribosome binding site, a transcription termination site, a selection marker, or a combination thereof. The host cell may express the gene operably linked to the promoter, for example, under anaerobic conditions. For example, the gene may be highly expressed under aerobic conditions and may also maintain a relatively high level of gene expression under anaerobic conditions. Under aerobic conditions, an expression level of the gene may be 0.5 times or higher, for example, 1.0 times or higher, 1.2 times or higher, 1.3 times or higher, or 1.4 times or higher than the expression level of the same gene operably linked to a tac promoter, based on an mRNA or protein level.

Another aspect of the invention provides a method of expressing the target gene, the method including culturing the host cell including the recombinant polynucleotide including the promoter region comprising the nucleotide sequence of positions 421 to 450 of SEQ ID NO: 1, the nucleotide sequence of positions 248 to 273 of SEQ ID NO: 2, or the nucleotide sequence of SEQ ID NO: 3 and the target gene operably linked to the promoter region to express the target protein.

The method includes culturing the host cell so as to express the target gene and produce the target protein.

In the method, the host cell may be a cell of the family Acetobacteraceae. The target gene may encode a protein involved in cellulose production, for example, synthesis. The gene encoding the target product may be a gene encoding permease, glucose kinase (GLK), phosphoglucomutase (PGM), UDP-glucose pyrophosphorylase (UGP), or cellulose synthase (CS). The gene encoding the target product may be an E. coli (Ec) glcP gene, a Zymomonas mobilis (Zm) glk gene, a Komagataeibacter xylinus (Kx) pgm gene, an E. coli (Ec) galU gene, a Xanthomonas campestris (Xc) UGP gene, or a Komagataeibacter xylinus (Kx) bcsABCD gene.

The method may be, for example, a method of culturing the vector-introduced host cells to produce a final product in a biosynthetic pathway involving the protein encoded by the target gene. The target gene may be, for example, involved in production, for example, synthesis of a product selected from the group consisting of cellulose, L-amino acids, lactic acid, acetic acid, succinic acid, and combinations thereof. Therefore, the method may be used to produce the final product of the gene, for example, a product selected from the group consisting of cellulose, L-amino acids, lactic acid, acetic acid, succinic acid, and combinations thereof under aerobic or anaerobic conditions. In the method, the product may be, for example, produced under aerobic conditions, and maintained under anaerobic conditions. The host cell may be, for example, K. xylinus KCCM 41431 that is introduced with a vector, in which the gene involved in the production, for example, synthesis of the product is operably linked to the promoter having the nucleotide sequence of SEQ ID NO: 1, 2, or 3 or a variant thereof.

The culturing of the host cell may be performed according to general methods known in the art. A medium used for the culturing may include a sugar source, for example, sugar and carbohydrate (e.g., glucose, saccharose, lactose, fructose, maltose, and starch), oil and fat, (e.g., soybean oil, sunflower oil, castor oil, and coconut oil), a fatty acid, (e.g., palmitic acid, stearic acid, and linolenic acid), an alcohol, (e.g., glycerol and ethanol), and an organic acid, (e.g., acetic acid), singly or in a mixture. The medium may include as a nitrogen source, for example, peptone, yeast extract, meat extract, malt extract, corn steep liquor, soy meal and urea, or an inorganic compound, e.g., ammonium sulfate, ammonium chloride, ammonium phosphate, ammonium carbonate and ammonium nitrate, singly or in a mixture. The medium may include as a phosphorous source, for example, potassium dihydrogen phosphate, dipotassium hydrogen phosphate, or a corresponding sodium-containing salt thereof. The medium may include, for example, a metal salt, e.g., magnesium sulfate or iron sulfate, which is essential for growth. Also, in the culturing, substances essential for growth, such as amino acids and vitamins, or suitable precursors may be added to the culture. Those components may be added to the culture in a proper manner, for example, in a batch or continuous manner during the culturing.

The culturing may be performed under aerobic conditions.

Reference will now be made in detail to exemplary embodiments, examples of which are illustrated in the accompanying drawings, wherein like reference numerals refer to like elements throughout. In this regard, the present exemplary embodiments may have different forms and should not be construed as being limited to the descriptions set forth herein. Accordingly, the exemplary embodiments are merely described below, by referring to the FIGURES, to explain aspects. As used herein, the term “and/or” includes any and all combinations of one or more of the associated listed items.

Hereinafter, the present invention will be described in more detail with reference to Examples. However, these Examples are for illustrative purposes only, and the scope of the invention is not intended to be limited by these Examples.

### Example 1: Exploration of Promoters and Identification of P1, P2, and P3

In this Example, three kinds of promoters which may be utilized in gene overexpression were explored in acetic acid bacteria, especially, microbial cellulose-producing bacteria of the genus Komagataibacter. All of these promoters were derived from a genome of Komagataibacter xylinus KCCM 41431 which is a natural microbial cellulose-producing bacterium.

(1) Exploration of Promoter

The genomic DNA of K. xylinus KCCM 41431 was extracted, and partially digested with Sau3AI. Of digestion products, DNA fragments having a size of 0.5 to 1.5 kb were extracted from a 1% agarose gel. Each of the extracted DNA fragments was ligated to a promoter exploration vector, pTSaP having a nucleotide sequence of SEQ ID NO: 4, which was digested with BgIII. These vectors were transformed into E. coli Top10 strain, and then plated on Luria Delbruck (LB) solid media containing 10 ug/ml of tetracycline and 5 ug/ml of chloramphenicol. Plasmids were isolated from pooled colonies, and K. xylinus KCCM 41431 was transformed with each of the plasmid by electroporation, and then plated on a Hestrin-Schramm (HS: 20 g/l of glucose, 5 g/l of yeast extract, 5 g/l of polypeptone, 2.7 g/l of Na₂PO₄ and 1.15 g/l of citric acid) solid medium containing 5 ug/ml tetracycline to obtain colonies. In pTSaP, the inserted genomic DNA and a reporter gene, chloramphenicol acetyltransferase (cat) gene were operably linked. The promoter exploration vector pTSaP was a custom-made vector. In pTSaP, a replication origin pSa ori which allows initiation of replication in cells of the genus Komagataibacter, a tetracycline resistance gene, an E. coli replication origin pUC ori, and the reporter gene cat are operably linked to a transcription terminator.

K. xylinus KCCM 41431 colonies thus obtained were passaged in HS solid media containing 5 ug/ml of tetracycline and 120 ug/ml of chloramphenicol, respectively. Plasmids were isolated from K. xylinus KCCM 41431 colonies which were successfully passaged by culturing at 30° C. for 48 hours or longer. Then, the plasmids were used as a template and polynucleotides of SEQ ID NOS: 5 and 6 were used as primers to perform sequencing.

As a result of the sequencing, a sequence of DNA cloned into the BgIII site of the pTSaP vector was revealed, and the obtained promoters were designated as P1, P2, and P3, respectively. The P1, P2, and P3 promoters have nucleotide sequences of SEQ ID NOS: 1, 2, and 3, respectively. Transcription start sites of these sequences were predicted by a promoter prediction program (Genome2D webserver for analysis and visualization of bacterial genomes and transcriptome data, de Jong et al. BMC Genomics 2012, 13: 299). As a result, the predicted promoter region of SEQ ID NO: 1 was a nucleotide sequence of positions 421 to 450, ‘TATAATGCATTCTGATATTTTGTTGTTAT’ (SEQ ID NO: 8), and the transcription start site was T at position 455. The predicted promoter region of SEQ ID NO: 2 was a nucleotide sequence of positions 248 to 273, ‘TTAAATTTTTCATACTTATTAATGTAAAAT’ (SEQ ID NO: 9), and the transcription start site was T at position 283.

(2) Evaluation of Promoter Strength

The promoter activity of the inserted genomic DNA was determined by measuring expression strength of the reporter gene, cat, i.e., strength of CAT activity. With regard to control groups, pTSaP introduced without the genomic DNA was used as a negative control group, and pTSaP containing a generally used tac promoter (SEQ ID NO: 7) was used as a positive control group.

CAT activity was determined by a CAT assay that measures acetylated chloramphenicol, and the CAT assay was performed as follows. Acetyl-CoA was reacted with chloramphenicol in the presence of CAT enzyme and 5,5′-dithio-bis (2-nitrobenzoic acid (DTNB) to produce acetyl-chloramphenicol and CoA. In this regard, CoA was reacted with DTNB to be converted into 5-thio-2-nitrobenzoate (TNB) having absorbance at 412 nm. That is, K. xylinus colonies obtained in section (1) and control groups were cultured in HS liquid media containing tetracycline (5 ug/ml) and cellulose (0.5%, Sigma C2730) at 30° C. for 24 hours under stirring at 220 rpm. The bacteria were harvested and suspended in PBS buffer, and then disrupted by sonication, followed by centrifugation. A supernatant was collected and a crude protein was obtained. Next, 10 ug of the crude protein per 1 ml of a reaction solution was mixed and reacted with acetyl-CoA (200 ug/mL), chloramphenicol (100 ug/mL), and DTNB (50 mg/mL), and absorbance at 412 nm was measured over time. The measured absorbance was applied to the following equation to calculate the activity.

activity (units/ml enzyme)=(Δ412 nm/min test−Δ412 nm/min Blank)(df)/(0.0136)

(df: dilution factor)

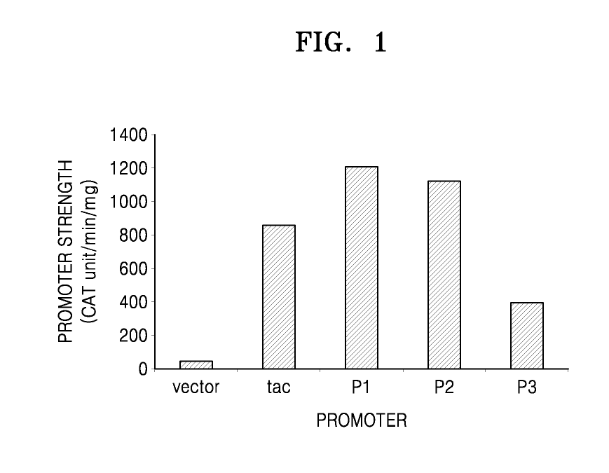

FIG. 1 shows the result of CAT assay. In FIG. 1, vector (control group), tac, P1, P2, and P3 on the horizontal axis represent the pTSaP vector introduced without the genomic DNA, the pTSaP vector introduced with the tac promoter, and the pTSaP vector introduced with the promoter of SEQ ID NO: 1, 2, or 3, respectively. As shown in FIG. 1, P1, P2, and P3 showed strength 1.41 times, 1.31 times, and 0.46 times higher than that of the positive control group, tac promoter, respectively. P1, P2, and P3 also showed marked expression-improving effects, compared to the negative control group.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. An isolated recombinant polynucleotide comprising a promoter region having a nucleotide sequence comprising positions 421 to 450 of SEQ ID NO: 1, a nucleotide sequence comprising positions 248 to 273 of SEQ ID NO: 2, or a nucleotide sequence comprising SEQ ID NO: 3, and a polynucleotide sequence encoding a target protein operably linked to the promoter region.

2. The polynucleotide of claim 1, wherein the promoter comprises SEQ ID NO: 1, 2, or 3.

3. The polynucleotide of claim 1, wherein the polynucleotide is a vector.

4. The polynucleotide of claim 1, wherein the target protein is a protein involved in cellulose production.

5. The polynucleotide of claim 4, wherein the target protein is permease, glucose kinase (GLK), phosphoglucomutase (PGM), UDP-glucose pyrophosphorylase (UGP), or cellulose synthase (CS).

6. A host cell comprising the recombinant polynucleotide of claim 1.

7. The host cell of claim 6, wherein the host cell is an acetic acid-producing bacterium.

8. The host cell of claim 6, wherein the host cell is a cell of the family Acetobacteraceae.

9. The host cell of claim 8, wherein the cell of the family Acetobacteraceae is a cell of the genus Komagataibacter, the genus gluconacetobacter, the genus acetobacter, or the genus gluconobacter.

10. The host cell of claim 6, wherein the host cell is a Komagataibacter xylinus cell.

11. The host cell of claim 6, wherein the recombinant polynucleotide is a vector.

12. The host cell of claim 6, wherein the recombinant polynucleotide comprises a polynucleotide sequence encoding a target protein encodes a target protein.

13. The host cell of claim 6, wherein the recombinant polynucleotide comprises a polynucleotide sequence encoding a target protein involved in cellulose production.

14. The host cell of claim 7, wherein the target protein is permease, glucose kinase (GLK), phosphoglucomutase (PGM), UDP-glucose pyrophosphorylase (UGP), or cellulose synthase (CS).

15. A method of producing a target protein, the method comprising culturing a host cell comprising a recombinant polynucleotide of claim 1 to express the target protein.

16. The method of claim 15, wherein the host cell is a cell of the family Acetobacteraceae.

17. The method of claim 15, wherein the target gene encodes a protein involved in cellulose production.

18. The method of claim 17, wherein the target protein is permease, glucose kinase (GLK), phosphoglucomutase (PGM), UDP-glucose pyrophosphorylase (UGP), or cellulose synthase (CS).

19. The method of claim 15, wherein the culturing is performed under aerobic conditions.


SEQUENCE LISTING (9 sequences)

SEQ ID NO: 1 - DNA, 1141 bp, Komagataibacter xylinus
  misc_feature (1)..(1141): KCCM 41431
gatcgcgggcacgcgctgcattgaccgccagcgcggcggcagccgttttggcgcccaccgcaccggacttgcgctggctgaccgggtgagcgggcgcgcagccgccctgtccagcacggggcagcagaaggcgctgctgctgggcatcgtgctgtcgcatgcgcgcatactcgcggcatgccgggggcaggtgcccatgctcctgctcgatgagccgctggtgcatctggacgaagcccgcaggcaggccctgttccgcgccgtgggccgcatgcgcacgggcgtgttcctgaccggaacggacgccgaacagttcgcccccctgcggggccatgccgcattcgaggcgccaggggcgggtaatcttgcccatgaggcctgatttgcgtggggaatgctggttccgacgggcggttggggctataatgcattctgatattttgttgttatccgctgtggagcatctgccggcatgtccgatcaatccagtcccgatcagaaacacgatgccgaggccaagggcacagtagcgcccgcgcctgattatgatgaggcatccatctcggtgctgcgggggctggatgcggtgcgcaagcgccccggcatgtatattggcgataccgatgacggctcgggcctgcaccacatggccttcgaaatcattgataacgcggtggatgaggcgcaggccggtttcgccaccggctgcgtcgtcaccctcaatggcgatggcagcgtgaccgtgcgcgatgacgggcgcggcattcccaccggcatgcaccatgaggaaggggtgagtgcggcggaagtcgtgctgaccaagctgcatgcgggcggcaagttcaaccagaattcctacaaggtttccggtggcctgcatggcgtgggcgctgcggtggtcaatgctttgtccgaatggatggaagtgcgcatctggcgcgatggcaaggagcatgtgatccgctttcagggcggcgagcgtgatgaggcgctgcgcgtggtgggcgaaagtgctgagccgcgcggcacgcaggtcacattcaagcccagtgccaagaccttcgccaaggtggagttcgagttcccgattctcgagcgccgcctgcgcgaactggccttcctcaattccgggctcaggatc

SEQ ID NO: 2 - DNA, 1058 bp, Komagataibacter xylinus
  misc_feature (1)..(1058): KCCM 41431
gatcggtccacgtatcaataaactgtgcggactggtagagggccatcccgttctccattgtttttcatcggctggctgggctgttaaataacctttagcatgcatcttatgtggcggcacatccatcccccatgcagcatgctataatcctgtgctttttccccacctgtgggtattgcttcccatgcggggcaggtagattatttatgagggcatctgtcacaagccaacatcttttcagtacagaattaaatttttcatacttattaatgtaaaatgtaatttatattcctgttttatccatattgaaattattggatgtagaaaataacaaatttataaatagcataacagggttcgttttataggaaaatattcattgaaacgttttgcgaaaataacgtaacgattaaaaaacaataaaagttttttcaatgcagctttgtcaaaaaaaacttatcggatactggcagttgattgaaggggttgttcacaaaacccgccattatcccgtttcctgcctgcctttggcgtgccgggtagcatgatgcggacagcatgtaactgaaaggactgtctttcagtctggagaacgaagcgtcagcgactggtcaactgtttgtgtcacaatggcattcagcgcgccatgtgctgctgtccattgaaagggatcggcatcgttattgaaggtcgggtcaagctggtcgccccgtaccgtgccataggcctgaaaatacatgtcgggccgggcggggatggtgtccatcgtccatgtccagtcatcggacaggagagctgtagcgcctacctgcgcgcaggggcgcgtgggtgacagggtcacggctgccgtcgtcatgagggctgcctgcgggcggtggtagcggctgtctccgccaatatcttccatctgcaggtcgcggcggcgcgcttcctcggcgggcgggatgttgcgccagccatcgccatcggggcctgcgtgggcgcggtcgagcgggtccgatgcagggccaaaggcgtggcctgcgtaattgagcgcggtgcctgcgggcacatggatc

SEQ ID NO: 3 - DNA, 535 bp, Komagataibacter xylinus
  misc_feature (1)..(535): KCCM 41431
aacttcggcggcgcccgagcgtgaacagcacgggctgaccaacctgtgcgcgcgcggcggctacgtcctggcggaagccgaagggacgcggcaggtcacgctggtcgccacggggcacgaggcgatactggcgctggcggcacgcaaactgttgaaggacgcaggggttgcggcggctgtcgtatcccttccatgctgggaactgttcgccgcgcaaaaaatgacgtatcgtgccgccgtgctgggaacggcaccccggatcggcattgaagccgcgtcagggtttggatgggaacgctggcttgggacagacgggctgtttgttggcattgacgggttcgggacggccgccccggaccagccggacagcgcgactgacatcacgccggaacggatctgccgcgacgcgctgcgtctggtccgtcccctgtccgataccctgactgaaccggcgggaggaaacggcgcgccgcccgggatgacatcggccgatgtcagtgtgtgaaatgtcagaccttacggagaaaataagaaa

SEQ ID NO: 4 - DNA, 4843 bp, Artificial Sequence
  Synthetic pTSaP promoter screening vector
aagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagcatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatttttaaggcagttattggtgcccttaaacgcctggttgctacgcctgaataagtgataataagcggatgaatggcagaaattcgaattcagccagcaagacagcgatagagggtagttatccacgtgaaaccgctaatgccccgcaaagccttgattcacggggctttccggcccgctccaaaaactatccacgtgaaatcgctaatcagggtacgtgaaatcgctaatcggagtacgtgaaatcgctaataaggtcacgtgaaatcgctaatcaaaaaggcacgtgagaacgctaatagccctttcagatcaacagcttgcaaacacccctcgctccggcaagtagttacagcaagtagtatgttcaattagcttttcaattatgaatatatatatcaattattggtcgcccttggcttgtggacaatgcgctacgcgcaccggctccgcccgtggacaaccgcaagcggttgcccaccgtcgagcgccagcgcctttgcccacaacccggcggccggccgcaacagatcgttttataaatttttttttttgaaaaagaaaaagcccgaaaggcggcaacctctcgggcttctggatttccgatcacctgtaaagtgggaccacatgctgaactccctatcactgcatgagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctgcaggtcgactctagagatctaacttcggcggcgcccgagcgtgaacagcacgggctgaccaacctgtgcgcgcgcggcggctacgtcctggcggaagccgaagggacgcggcaggtcacgctggtcgccacggggcacgaggcgatactggcgctggcggcacgcaaactgttgaaggacgcaggggttgcggcggctgtcgtatcccttccatgctgggaactgttcgccgcgcaaaaaatgacgtatcgtgccgccgtgctgggaacggcaccccggatcggcattgaagccgcgtcagggtttggatgggaacgctggcttgggacagacgggctgtttgttggcattgacgggttcgggacggccgccccggaccagccggacagcgcgactgacatcacgccggaacggatctgccgcgacgcgctgcgtctggtccgtcccctgtccgataccctgactgaaccggcgggaggaaacggcgcgccgcccgggatgacatcggccgatgtcagtgtgtgaagatctcccgggtaccgagctctctagaaagaaggagggacgagctattgatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggttgctacgcctgaataagtgataataagcggatgaatggcagaaattcgtcgaggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttcctgtcg

SEQ ID NO: 5 - DNA, 24 bp, Artificial Sequence
  Synthetic SeqF primer
atgttctttacgatgccattggga

SEQ ID NO: 6 - DNA, 22 bp, Artificial Sequence
  Synthetic SeqR primer
tctcctgagtaggacaaatccg

SEQ ID NO: 7 - DNA, 188 bp, Artificial Sequence
  Synthetic tac promoter
ggctgtgcaggtcgtaaatcactgcataattcgtgtcgctcaaggcgcactcccgttctggataatgttttttgcgccgacatcataacggttctggcaaatattctgaaatgagctgttgacaattaatcatcggctcgtataatgtgtggaattgtgagcggataacaatttcacacaggaaacat

SEQ ID NO: 8 - DNA, 29 bp, Artificial Sequence
  Synthetic P1 promoter
tataatgcattctgatattttgttgttat

SEQ ID NO: 9 - DNA, 30 bp, Artificial Sequence
  Synthetic P2 promoter
ttaaatttttcatacttattaatgtaaaat